(19) United States
(12) Patent Application Publication
(10) Pub. No.: US 2016/0287218 A1
(43) Pub. Date: Oct. 6, 2016

(54) ULTRASOUND IMAGE MARKER

(71) Applicant: Ruben Quintero, Miami, FL (US)

(72) Inventor: Ruben Quintero, Miami, FL (US)

(21) Appl. No.: 15/087,688

(22) Filed: Mar. 31, 2016

Related U.S. Application Data
(60) Provisional application No. 62/140,875, filed on Mar. 31, 2015.

(51) Int. Cl. A61B 8/08

(52) U.S. Cl. CPC A61B 8/5292

(57) ABSTRACT

The method in an ultrasound system for marking an ultrasound medical image so as to indicate its orientation is disclosed. The method comprises receiving a signal from a compass on an ultrasonic probe during use on a patient, wherein the signal is produced in relation to a fixed reference point, receiving a signal from an accelerometer on the ultrasonic probe during use on the patient, wherein the signal is produced in relation to the fixed reference point, processing the two signals to determine the orientation of the patient, or portion of the patient, during use of the system on the patient, generating an ultrasound medical image based on imaging data received from the ultrasonic probe during use on the patient, and placing a marker on the ultrasound medical image, wherein said marker corresponds to the orientation of the patient, or portion of the patient.

### CROSS-REFERENCE TO RELATED APPLICATIONS

This application claims priority to provisional application No. 62/140,875 filed Mar. 31, 2015 and entitled ULTRASOUND IMAGE MARKER. The subject matter of application Ser. No. 62/140,875 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The disclosed embodiments relate to the field of medical imaging and more specifically to processes for determining the orientation of a medical image.

### BACKGROUND

Medical images include x-rays, ultrasounds, MRIs, CAT scans, etc. Ultrasonic measurements are performed using a device, a transducer, also referred to as a probe, which is placed over the patient's skin. The transducer or probe serves as both the emitter and receiver of ultrasonic signals. The emitted signal (outgoing signal) has a certain frequency, amplitude and shape, to allow for proper imaging of the organ or organs under investigation. The received signal (incoming signal) is processed and subject to a waveform analysis algorithm to create the image of the organ or organs under investigation. It is very important from a diagnostic point of view that the image of the organ or organs is properly identified relative to other organ or organs of the body—i.e., its orientation.

One of the problems associated with viewing medical images is that often the viewer has no idea of the orientation of the image being viewed. Because medical images can often appear like a series of undistinguishable blobs and shapes with little or no indicator as to the orientation of the patient during imaging, the viewer is often left to guessing how the image should be viewed and regarded. It is important for the viewer to know whether he is viewing a frontal view, a side view or a rear view, because this data is used by the viewer to determine what the viewer is viewing, what a normal image should look like, and how to discern abnormalities or other problems with the patient. Further, knowing whether the medical image is a transverse, coronal or sagittal plan is also important for the viewer, because the viewer uses this data to identify what the viewer is viewing and to identify issues or abnormalities in the patient.

Therefore, there exists a need for improvements over the prior art, and more particularly for a more efficient way of identifying the orientation of an ultrasound image.

### SUMMARY

In one embodiment, a method in an ultrasound system for marking an ultrasound medical image so as to indicate its orientation is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

The method in an ultrasound system for marking an ultrasound medical image so as to indicate its orientation is disclosed. The method comprises receiving a signal from a compass on an ultrasonic probe during use on a patient, wherein the signal is produced in relation to a fixed reference point, receiving a signal from an accelerometer on the ultrasonic probe during use on the patient, wherein the signal is produced in relation to the fixed reference point, processing the two signals to determine the orientation of the patient, or portion of the patient, during use of the system on the patient, generating an ultrasound medical image based on imaging data received from the ultrasonic probe during use on the patient, and placing a marker on the ultrasound medical image, wherein said marker corresponds to the orientation of the patient, or portion of the patient, during use of the system on the patient.

### BRIEF DESCRIPTION OF THE DRAWINGS

The accompanying drawings, which are incorporated in and constitute part of this specification, illustrate embodiments of the claimed subject matter and together with the description, serve to explain the principles of the invention. The embodiments illustrated herein are presently preferred, it being understood, however, that the invention is not limited to the precise arrangements and instrumentalities shown, wherein:

FIG. 1 is an illustration of an ultrasound system with a marker used to define orientation, according to one embodiment.

FIG. 2 is an illustration of an ultrasound image, according to one embodiment.

FIG. 3 is an illustration of an ultrasound image, according to one embodiment.

### DETAILED DESCRIPTION

The disclosed embodiments improve upon the problems with the prior art by providing a process for marking or otherwise indicating on a medical image the orientation of the image or the patient (or portion thereof) in the image. The claimed system is a necessary add-on to the transducer in order to overcome the lack of relative positioning (the reference) that the current transducers currently use. The claimed system refers to the device and to the transmission protocol that allows this reference to be placed in the ultrasound image. Its application covers the entire range of diagnostic sonography (ultrasonography) used to visualize internal body structures including tendons, muscles, joints, vessels and internal organs for possible pathology or lesions. The application of the claimed system in the practice of examining pregnant women using ultrasound, called obstetric sonography, is widely used and is also included herein.

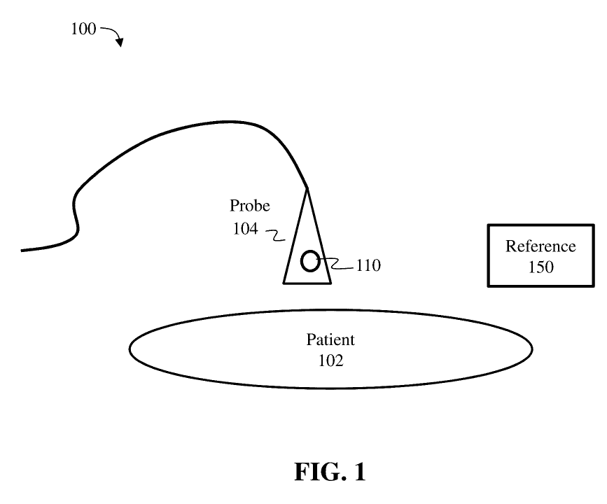

FIG. 1 is an illustration of an ultrasound system 100 with a reference or marker 150 used to define orientation, according to one embodiment. An ultrasound system is a system known in the art for using a diagnostic imaging technique based on the application of ultrasound. It is used to see internal body structures such as tendons, muscles, joints, vessels and internal organs. Its aim is often to find a source of a disease or to exclude any pathology. The practice of examining pregnant women using ultrasound is called obstetric ultrasound, and is widely used.

FIG. 1 indicates an arbitrary region in a person's body where the claimed system would apply. The region of the patient's body 102 investigated by the ultrasonic probe 104 is shown in the figure. The figure also shows a reference point or marker 150. The established methodology consists of placing the probe 104 over the person's skin so that the ultrasound waves can be propagated from the probe 104 into the organ or organs of the patient, through the tissues, and then reflected and received by the probe. The probe then transmits the imaging signal to the computing device portion of the ultrasound system. The data is then processed by the computing device portion of the ultrasound system, and the resulting data is visualized as an ultrasound medical image.

One variant is to place the reference point or marker 150 within the body of the probe. The product may consist of one or more subparts, such that at least one orientation of the probe or ultrasonic transducer is properly recorded. Variants of may include more parts as required for redundancy or to increase resolution and accuracy

A small real-time accelerometer and compass 110 may be attached to the ultrasonic probe 104. The device 110 may emit a real-time signal, referred to as the positioning signal, which may be transmitted in the same bundled transmission of the ultrasound image signal transmitted by the probe, either via wire or wireless. FIG. 1 shows a potential location of the item 110 within probe 104, though said device 110 may be located elsewhere, such as outside the probe 104. A variant of device 110 consists of a magnetometer or compass which transmits its relative position to a fixed reference that the user selects. A variant would consist of inclinometers which would depict in three dimensions the relative position of the transducer's reference position. The compass and inclinometers can work either independently or jointly, with data being transmitted either in sequence or in parallel to the signal transmitted by the ultrasonic transducer or probe.

Since the probe moves at a much slower frequency than the actual ultrasound outgoing signal, the transmission protocol can be such that the positioning signal is sent at a much lower frequency to the computing device portion of the ultrasound system. The power to the device 110 would come either from the same source that generates the ultrasonic pulses, or via a battery, or self-generated from the movement of the probe. The accelerometer and/or compass are connected via wired connections or via wireless connection to a transmitter device which may be located in the body of the ultrasound housing. The compass and accelerometer may transmit the following data to a processor (such as a processor in the computing device portion of the ultrasound system): relative positioning data and motion data; relative distance of the probe 104 to the reference 150 in all three dimensions, and in any system of units; latitude, longitude in any format, and in any system of units; acceleration of the device 110, in any format and in any system of units; and velocity of the device 110 in any system of units. The data processed or unprocessed is incorporated into the ultrasound image in order to provide any combination of the relative position and/or motions described above. The output of the processor is any and all relative positioning data and motion data.

Following is a suggested mode of operation of the claimed system: 1) an operator selects a reference point, such as 150 (this selection is either done via the probe or in the computing device portion of the ultrasound system), 2) an operator places the probe 104 with device 110 such that the probe 104 with device 110 is aligned to the reference 150; 3) the position of the probe 104 relative to the reference 150 is recorded; and 4) an operator performs diagnostic imaging using probe 104, which images will now have the relative position of the reference stamped throughout. Thus, at the outset, the operator identifies the probe position as the corresponding orientation relative to a body part (such as the frontal part of the head of the mother or patient). The operator may then identify a second and third position (the back part of the heard of the mother, for example, and the left side of the abdomen of the mother, for example). Once the reference orientations are obtained, the compass and inclinometer data can be immediately shown in the corresponding new reference system.

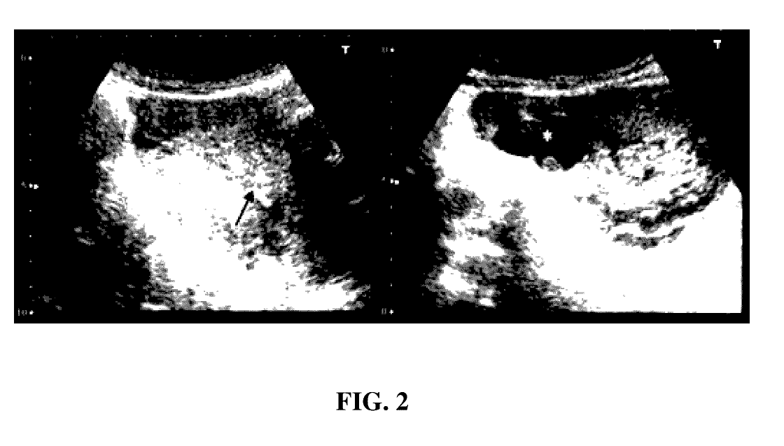

FIG. 2 is an illustration of an ultrasound image, according to one embodiment. FIG. 2 shows an ultrasonic image of the uterus of a patient after evacuation of the non-viable intrauterine pregnancy. As shown in FIG. 2, all ultrasound images to date lack a reference point that shows orientation. In the example shown, the viewer cannot know the orientation of the patient when viewing the image, and therefore the viewer must infer where the head of the patient was located when the image was taken. Without information about the orientation of the patient when the image was taken, serious misdiagnosis may occur.

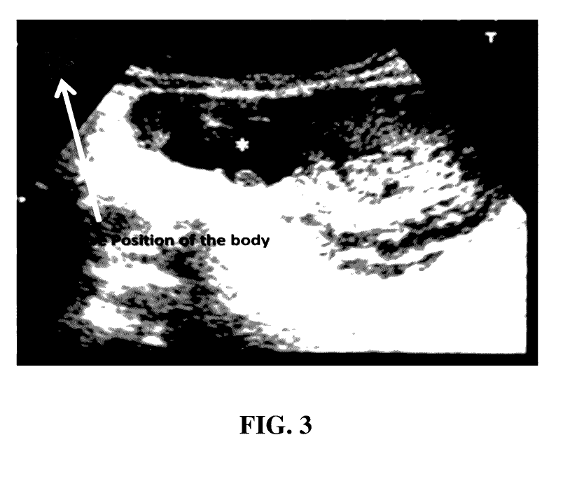

FIG. 3 is an illustration of an ultrasound image, according to one embodiment. The claimed system would add orientation reference information into the image, as shown in FIG. 3. FIG. 3 shows an ultrasonic image of the uterus after evacuation of the non-viable intrauterine pregnancy. FIG. 3 also shows an orientation reference or indicator in the top left corner. The orientation reference or indicator can be an image showing the position of the body, a part of the body, or other type of information that can be shown in the ultrasound image. Also, the orientation reference or indicator can be static or dynamic. A static reference is entered when the probe's position with respect to the reference will not change. A dynamic reference indicates that as the image of the reference moves as the probe moves in order to always have an accurate representation of the ultrasound image with respect to the reference.

Although specific embodiments have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the invention.

We claim:

1. A method in an ultrasound system for marking an ultrasound medical image so as to indicate its orientation, the method comprising:
receiving a signal from a compass on an ultrasonic probe during use on a patient, wherein the signal is produced in relation to a fixed reference point;
receiving a signal from an accelerometer on the ultrasonic probe during use on the patient, wherein the signal is produced in relation to the fixed reference point;
processing the two signals to determine the orientation of the patient, or portion of the patient, during use of the system on the patient;
generating an ultrasound medical image based on imaging data received from the ultrasonic probe during use on the patient; and
placing a marker on the ultrasound medical image, wherein said marker corresponds to the orientation of the patient, or portion of the patient, during use of the system on the patient.
